SEQUENCE LISTING (1 sequence)

SEQ ID NO: 1 - protein, 418 aa, Homo sapiens [sequence omitted]

(19) United States
(12) Patent Application Publication
(10) Pub. No.: US 2010/0255074 A1
(43) Pub. Date: Oct. 7, 2010

(54) TRANSMEMBRANE PROSTATIC ACID PHOSPHATASE

(75) Inventor: Pirkko Vihko, Helsinki (FI)

Correspondence Address: VOLPE AND KOENIG, P.C., UNITED PLAZA, 30 SOUTH 17TH STREET, PHILADELPHIA, PA, 19103, US

(73) Assignee: CHEMPATH OY, Helsinki (FI)

(21) Appl. No.: 12/665,232

(22) PCT Filed: Jun. 19, 2008
(86) PCT No.: PCT/FI08/50377

§ 371 Date: May 20, 2010

(30) Foreign Application Priority Data
Jun. 19, 2007 (FI) 20075466

(51) Int. Cl. A61K 9/127; A61K 38/46; A61K 39/395; A61P 35/00

(52) U.S. Cl. 424/450; 424/94.6; 424/134.1

(57) ABSTRACT

The present invention relates to a novel transmembrane prostatic acid phosphatase (TM-PAP) protein or the C-terminal part thereof, nucleic acid molecules encoding said protein, vectors containing said nucleic acid molecules and host cells expressing said proteins. The present invention relates also to pharmaceutical compositions containing TM-PAP or the C-terminal part thereof and methods for using thereof in therapy and diagnostics. The present invention also relates to methods utilizing a transmembrane prostatic acid phosphatase knockout/knockdown non-human animal model and uses thereof.

### FIELD OF THE INVENTION

The present invention relates to a novel transmembrane prostatic acid phosphatase (TM-PAP) protein, nucleic acid molecules encoding said protein, vectors containing said nucleic acid molecules and host cells expressing said proteins. The present invention relates also to pharmaceutical compositions containing TM-PAP and methods for using thereof in therapy and diagnostics. The present invention also relates to methods utilizing a transmembrane prostatic acid phosphatase knockout/knockdown non-human animal model.

### BACKGROUND OF THE INVENTION

Prostate cancer is the most common cancer in men and the second leading cause of cancer death in the Western countries. Although chemotherapy treatments have shown survival benefit for hormone-refractory prostate cancer, new and more effective therapies are needed. A novel approach refers to the activation of the immune system using an antigen loaded in antigen-presenting cells (Srivastava P K. Therapeutic cancer vaccines. Curr Opin Immunol 2006; 18: 201-5). Some of the new ongoing vaccine therapies in trial for prostate cancer treatment are based on the essential restriction of PAP (ACPP, EC 3.1.3.2) expression in prostate (Lin A M, Hershberg R M, Small E J. Immunotherapy for prostate cancer using prostatic acid phosphatase loaded antigen presenting cells. Urol Oncol 2006; 24: 434-41). US2006/0294615 discloses a therapeutic method for treating a mammalian prostate carcinoma comprising the step of administering a therapeutically effective amount of cellular PAP protein to the carcinoma. This and all the other publications referred to herein are incorporated by reference.

There are two forms of PAP, secretory and non-secretory, with different isoelectric points and molecular weights (Vihko P. Human prostatic acid phosphatases: purification of a minor enzyme and comparisons of the enzymes. Invest Urol 1979; 16: 349-52). Only mRNA encoding secretory form is described so far (Vihko P, Virkkunen P, Henttu P, et al. Molecular cloning and sequence analysis of cDNA encoding human prostatic acid phosphatase. FEBS Lett 1988; 236: 275-81). It is suggested to encode also so-called cellular form (Veeramani S, Yuan T C, Chen S J, et al. Cellular prostatic acid phosphatase: a protein tyrosine phosphatase involved in androgen-independent proliferation of prostate cancer. Endocr Relat Cancer 2005; 12: 805-22). Cellular PAP has not been cloned.

It is claimed that PAP has growth-suppressing effect and it is due to its cellular protein tyrosine phosphatase activity. Within cells the activity of PAP is lower in prostate carcinomas than in normal prostates and both PAP mRNA and protein levels are decreased or absent in prostate carcinoma tissue (Hakalahti L, Vihko P, Henttu P, et al. Evaluation of PAP and PSA gene expression in prostatic hyperplasia and prostatic carcinoma using northern-blot analyses, in situ hybridization and immunohistochemical stainings with monoclonal and bispecific antibodies. Int J Cancer 1993; 55: 590-7). U.S. Pat. No. 7,094,533 discloses methods for diagnosing androgen-insensitive prostate carcinomas comprising the step of determining the expression of cellular PAP protein in the prostate carcinoma, a decrease in the expression being indicative of the androgen insensitive nature of the carcinoma.

New results found by the present inventor show that PAP has at least two splicing variants encoding a secretory form and a type I transmembrane (TM) protein (Quintero et al., 2007, Cancer Res 67, 6549-6554), which is in vesicles and membranes and is widely expressed in many non-prostatic cells and tissues.

According to the new results, it is important to highlight the fact that the expression of PAP is not exclusive to prostatic tissue, and this issue has to be considered for the evaluation of unwanted side effects of PAP-based immunotherapy. The present invention suggests TM-PAP to have still unrevealed physiological prostatic and non-prostatic functions.

### SUMMARY OF THE INVENTION

Prostatic acid phosphatase (PAP) is currently evaluated as a target for vaccine immunotherapy of prostate cancer. This is based on the previous knowledge about secretory PAP and its high prostatic expression. Herein a novel PAP spliced variant mRNA encoding a type I transmembrane (TM) protein with the extracellular N-terminal phosphatase activity and the C-terminal endosomal/lysosomal targeting signal (Yxxcφ) is described. This TM-PAP is widely expressed in non-prostatic tissues like brain, brown adipose tissue, kidney, liver, lung, muscle, placenta, salivary gland, spleen, thyroid and thymus. TM-PAP is also expressed in fibroblasts, Schwann and LNCaP cells, but not in PC-3 cells. In well-differentiated human prostate cancer tissue specimens the expression of secretory PAP, but not TM-PAP, is significantly decreased. TM-PAP is localized in plasma membrane-endosomal-lysosomal pathway and is co-localized with the lipid raft marker flotillin-1. No cytosolic PAP is detected.

It is concluded that the wide expression of TM-PAP in, for instance, neuronal and muscle tissues, must be taken into account in the design of PAP-based therapy approaches.

Snapin is a protein associated in SNARE complex. “Yeast Two-Hybrid and co-immunoprecipitation was made as in Snapin interaction with the Exo70 subunit of the exocyst and modulates GLUT4 trafficking, J Biol Chem 283, 324-331, 2008” describes the role of Snapin in exocytose. The present inventor has discovered that TM-PAP interacts with Snapin therefore having a role in ecto- and exocytosis. This happens in all the cells wherein the Snapin is expressed. Snapin-mediated exocytosis is very important for the transport of sugar transport channels onto cell membrane. Also TM-PAP, when controlling Snapin, will also take part in this mechanism which controls the ion channels in prostate, kidneys and lungs. Also antigen presentation, immune response, inflammation response and autoimmune response are controlled with this mechanism. This is important for the applications of the present invention especially in prostate, nerve tissue and brains. For example, prostate cancer is a vesicle transport disorder.

The TM-PAP knockout/knockdown mouse described herein is a model animal for these disorders. The secreted PAP does not interact with Snapin because the interacting part is the intracellular part not found in secreted PAP. Snapin is an intracellular protein attached to plasma membrane. It was also found out in the present invention that when prostate cancer cells PC-3 were transfected with TM-PAP the growth of the cell was arrested. This does not happen if secreted PAP is used.

It is characteristic for the present invention what is disclosed in the independent claims. Some embodiments of the invention are disclosed in the dependent claims.

One aspect of the present invention relates to a novel prostatic acid phosphatase protein having a transmembrane domain and endosomal/lysosomal targeting signal in the C-terminus, or the C-terminal part thereof.

Another aspect of the present invention relates to an isolated DNA molecule encoding said transmembrane prostatic acid phosphatase protein or the C-terminal part thereof.

Still another aspect of the present invention relates to a nucleotide vector containing said DNA molecule.

Still another aspect of the present invention relates to a recombinant host cell containing said nucleotide vector.

Still another aspect of the present invention relates to a pharmaceutical composition containing said transmembrane prostatic acid phosphatase protein or the C-terminal part thereof.

Still another aspect of the present invention relates to said transmembrane prostatic acid phosphatase protein or the C-terminal part thereof for use as medicament.

Still another aspect of the present invention relates to use of said transmembrane prostatic acid phosphatase protein or the C-terminal part thereof for manufacturing medicament for treating disorders such as vesicle transport disorders, such as cancer, for example prostate cancer, prostatitis, respiratory or kidney diseases; inflammatory, immunodefence or autoimmune diseases, lymphoproliferative disorders such as leukemia, bone marrow proliferation, or metabolic disorders.

Still another aspect of the present invention relates to a method for delivering drugs, nanoparticles, imaging reagents or other molecules to cells using said transmembrane prostatic acid phosphatase protein or the C-terminal part thereof as a carrier.

Still another aspect of the present invention relates to a method for treating disorders such as vesicle transport disorders, such as cancer, for example prostate cancer, lymphoproliferative disorders such as leukemia, bone marrow proliferation, or metabolic disorders, by administering said transmembrane prostatic acid phosphatase protein or the C-terminal part thereof to a patient suffering from said disorder.

Still another aspect of the present invention relates to a method for diagnosing prostate cancer by determining the ratio of TM-PAP and secreted PAP in a tissue.

Still another aspect of the present invention relates to a non-human animal having a disruption in the transmembrane prostatic acid phosphatase gene or regulation thereon resulting in a decrease or absence of the activity or the level of prostatic acid phosphatase. The present inventor has previously described such knockout animal having a disruption in secreted PAP gene in WO06051172A1, which is incorporated herein by reference. At that time there was no knowledge about transmembrane PAP, but the concept and the uses of said animal model can be directly applied also to TM-PAP.

One aspect of the present invention relates to a method for testing and screening for a compound for an therapeutic effect, said method comprising the steps of administering said compound to a cell or a non-human animal having disruption in the transmembrane prostatic acid phosphatase gene or regulation thereof resulting in a decrease or absence of the activity or the level of prostatic acid phosphatase, and determining if said compound substantially restores the unbalanced phosphatidylinositol phosphate signaling pathway related to TM-PAP expression or activity on said cell or said animal, said restoring indicating said compound being therapeutically effective for treating disorders related to unbalanced phosphatidylinositol phosphate signaling pathway. The response of said restoring may be decrease in PI(4,5)P₂ accumulation or decrease in the level of PI(3)P, caused by the recovered PAP activity or level of expression. The term “substantially restoring” as used herein refers to such restoration or normalization of unbalanced PIP signaling pathway, either complete or partial, which has therapeutic value and effect. These methods may be used for investigating diseases and disorders related to TM-PAP, such as vesicle transport disorders, for example prostate hypertrophy, tumors or cancer. Such methods include for example testing and screening of a drug candidate compound or the like.

Another aspect of the current invention relates to methods for treating disorders related to unbalanced phosphatidylinositol phosphate cascade and/or signaling pathway related to transmembrane prostatic acid phosphatase resulting in PI(4,5)P₂ accumulation, increased level of PI(3)P or increased ratio of PI(4,5)P₂/PI(4)P, by administering a patient suffering said disorder a compound which increases the level of expression or activity of TM-PAP or by giving said patient gene therapy which increases the level or restores the activity of TM-PAP.

The present invention also utilizes a knockout/knockdown animal model wherein the transmembrane prostatic acid phosphatase gene in the genome of said animal has been disrupted resulting in a decrease in the activity or the level of transmembrane prostatic acid phosphatase. Said knockout/knockdown animal expresses a reduced level or activity of TM-PAP enzyme in certain cells or tissues or preferably does not express TM-PAP at all.

Also an isolated knockout/knockdown animal or plant cell, such as a prostate cell, may be used. Such cells may be cultured and used to investigate disorders related to TM-PAP and its function. The animals described above may be used as a source of said cells. In one embodiment said cell may be a human cell line, such as one derived from a human cancer cell line.

### BRIEF DESCRIPTION OF THE FIGURES

FIG. 1. Expression and splicing of PAP variants. (A) Total RNA from human, mouse and rat prostate tissue was analyzed for TM-PAP variant by RT-PCR using ATG and TGA containing primers or primer from 3′ side of the stop codon. (B) Exon-intron boundaries of the alternatively spliced intron and deduced amino acids of TM-PAP and secreted PAP. Upper case letters represent exon nucleotides (upper line) and amino acid residues (lower line), and lower case letters represent intron nucleotides of PAP (upper line). The length of spliced region is expressed as by in parentheses. The number of non-presented nucleotides in the last exon and amino acid residues in the C-terminus is marked in parentheses. Conserved 5′ and 3′ splice site nucleotides of introns are marked in bold. * marks a stop codon, (1)-(2) human, (3)-(4) mouse and (5)-(6) rat PAP, TM and secreted variants, respectively. Nucleotide sequence for rat TM-PAP variant was predicted from genome through our own Perl script programs using regular expressions. (C) Alignment of N- and C-termini of TM-PAP isoform with LAP. Identical residues are indicated by white letters in black boxes. Asterisks indicate residues required for lysosomal targeting (Yxxcφ). Alignments were performed using Clustal W (Thompson J D, Higgins D G, Gibson T J. CLUSTAL W: improving the sensitivity of progressive multiple sequence alignment through sequence weighting, position-specific gap penalties and weight matrix choice. Nucleic Acids Res 1994; 22: 4673-80) and ESPript (Gouet P, Courcelle E, Stuart D I, Metoz F. ESPript: analysis of multiple sequence alignments in PostScript. Bioinformatics 1999; 15: 305-8) for figures. Secondary structure elements for ESPript were obtained from PHD through PredictProtein server (Rost B, Yachdav G, Liu J. The PredictProtein Server. Nucleic Acids Res 2004; 32: W321-W326). Transmembrane prediction was performed with TMHMM server (Krogh A, Larsson B, von Hejne G, Sonnhammer E L. Predicting transmembrane protein topology with a hidden Markov model: Application to complete genomes. J Mol Biol 2001; 305: 567-80) and signal peptide was predicted using SignalP server (Bendtsen J D, Nielsen H, von Heijne G, Brunak S. Improved prediction of signal peptides: SignalP 3.0. J Mol Biol 2004; 340: 783-95). Gene Bank accession numbers: rat TM-PAP variant DQ826426, mouse TM-PAP variant NM_{—}207668, human TM-PAP variant BC007460, human secreted PAP variant NM_{—}001099, mouse LAP mRNA BC023343, human LAP mRNA BC093010 and human LPAP (lysophosphatidic acid phosphatase) mRNA AB031478. (D) Expression of TM-PAP in different mouse cells and tissues, and in human prostate cancer cells, and BPH and PC patient samples.

FIG. 2. PAP and BMP co-localization in human prostate cancer tissue. (A) PAP was localized in vesicles both in the basal and apical cytoplasm, and strong labeling was observed in the lumen of the glands. PAP showed an almost complete co-localization with BMP (PAP green, BMP red, nuclei in blue, co-localization in yellow). (B) PAP localized in the limiting and internal membranes of apical, electron-lucent vesicles (black arrow). PAP was also observed in the lumen of multi-vesicular endosomes. (C) In addition, PAP labeling was observed in the membranous structures in the lumen. PAP (10 nm gold) co-localized with BMP (5 nm gold, arrowheads). Some BMP labeling was also observed in the endosomes (right, arrowheads). AJ=adherent junction, L=lumen.

FIG. 3. PAP localization in LNCaP and SW10. (A) In LNCaP cells, PAP was detected in caveosome-like structures (˜100 nm, left), small endosome-like vesicles (˜65 nm, middle), and in lysosomes (right). In (A, left) gold labeling was enhanced by silver just enough to make the nanoparticles visible (indicated by arrows), to avoid any masking of the structures by the precipitate, whereas in (A, middle and right), a stronger signal was obtained by longer enhancement period. (B) PAP in isolated mouse Schwann cells. PAP was localized in the plasma membrane domains and filopodias.

FIG. 4. (A) TM-PAP localization in sarcolemma of human skeletal muscle detected by immunohistochemistry. (B) Localization of TM-PAP-GFP in plasma membrane and vesicles of PC-3 cells after transfection. Co-localization of PAP with different cell markers in LNCaP cells. (C) PAP co-localized with flotillin-1 in small vesicles and in the plasma membrane. (D) PAP co-localization with LAMP-2. White arrows show co-localization sites (yellow).

FIG. 5. The sequence alignment of the whole amino acid sequences of human, mouse and rat TM-PAPs and corresponding secreted PAPs. Signal peptide and transmembrane regions are marked as in FIG. 1.

FIG. 6. Figures (A)-(C) present the Snapin sequences (highlighted) which were found to interact with TM-PAP with yeast two-hybrid system.

### DETAILED DESCRIPTION OF THE INVENTION

### Novel PAP Isoform is Encoded by Alternative Splice Variant*

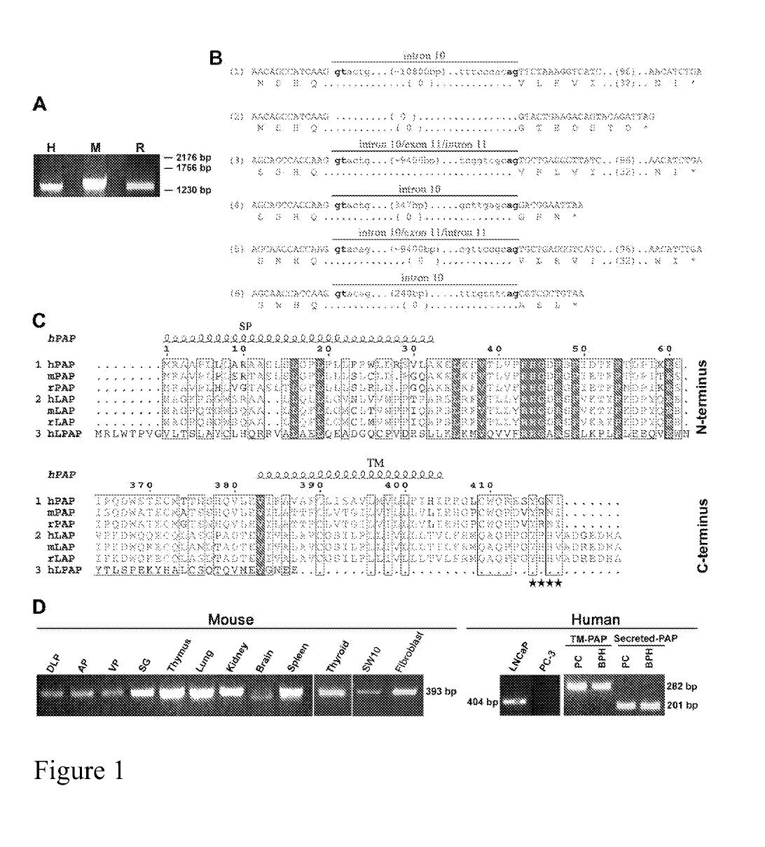

To generate human, mouse and rat TM-PAP variants by RT-PCR (FIG. 1A) total RNA was used. Rat and human PCR products were cloned and sequenced. Sequences were highly homologous with cDNA of mouse TM-PAP variant. The rat cDNA (GenBank accession number DQ826426), showed 91% and 81% identities with reported cDNA sequences of mouse (GeneBank accession number NM_{—}207668) and human PAP, respectively. Comparison of the exon-intron junction suggests that rat, mouse and human PAP variants are derived by alternative splicing. Position of the splicing of the 10^{th} intron in the rat, mouse and human gene is similar at the end of the 10^{th} exon. In the rat and mouse gene, the splicing of the 10^{th} intron results in the secreted variant mRNA and in human in the TM variant. The splicing of the 10^{th} intron, the 11^{th} exon and the 11^{th} intron in the rat and mouse gene yields the TM variant mRNA. In the human secreted variant mRNA, the open reading frame (ORF) continues over the splicing site until the stop codon (FIG. 1B).

The deduced 417-amino acid sequence showed 89% and 95% homologies with human and mouse PAP, respectively. The N-terminus of PAP has a 32 amino acids long signal peptide. The C-terminus of TM-PAP isoform contains the TM domain of 22 amino acid residues and an endosomal/lysosomal targeting Y[G/R]NI sequence separated by 10 amino acids from TM domain (FIG. 1C). The relative location of signal and TM peptides determines the topology of the TM-PAP isoform as a type I transmembrane protein. The present tyrosine signal Y[G/R]NI is similar to the lysosomal targeting sequence Yxxφ, where x can be any amino acid but tends to be hydrophilic and φ is a hydrophobic residue. As reviewed by Bonifacino and Traub (Bonifacino J S, Traub L M. Signals for sorting of transmembrane proteins to endosomes and lysosomes. Annu Rev Biochem 2003; 72: 395-447) this is evidence for lysosomal targeting. Moreover, the C-terminal residue of PAP, isoleucine, is the same as in LAMP-1, a type I TM protein known to be targeted to lysosomes. In conclusion, TM-PAP isoform can be efficiently internalized from plasma membrane due to the presence of the Yxxl motif and delivered to lysosomes through the indirect endosomal pathway as confirmed by our observations. Alignment of human, mouse and rat TM-PAP and lysosomal acid phosphatase (LAP) reveals similar TM domain, and lysosomal targeting sequence (FIG. 1C). Human LAP presents two cleavage sites for two different proteases. When C-terminal ends of hLAP and hPAP transmembrane isoform are compared, there is no identity of amino acids at the protease cleavage site or at the neighboring residues. Specificity requirements of these proteases suggest that TM-PAP is not a suitable substrate for them and it is possible that TM-PAP is recycling from lysosomes to plasma membrane.

### TM-PAP is Widely Expressed

Based on the contradictory statements present in the literature about the tissue-specific expression of PAP, the expression of this novel splicing variant in both human and mouse was analyzed. RT-PCR analyses showed that the TM-PAP variant was widely expressed in mouse tissues e.g. in prostate lobes, salivary gland, thymus, lung, kidney, brain, spleen, thyroid and mouse Schwann and fibroblast cells (FIG. 1D). Expression of the PAP secreted variant was clearly detected in salivary gland, thymus and thyroid (data not shown). Secretory and TM-PAP variants were present in LNCaP cells, but they were not expressed in PC-3 cells (FIG. 1D).

### The Ratio of PAP Isoforms is Changed in Prostate Cancer

Since immunohistochemically, PAP shows low expression in prostate carcinoma cells and tissues although in the circulation of prostate cancer patients its concentration may be elevated, PAP expression in prostate was further studied. Both PAP variants were present in human BPH and well-differentiated PC specimens (FIG. 1D). The abundance of PAP variants in BPH (n=27) and PC (n=19) specimens was determined using quantitative real-time PCR. Values of TM-PAP mRNA variant were (mean±SD) 0.5±0.26 in BPH and 0.45±0.34 in PC, and values of PAP mRNA secreted variant were 0.60±0.3 in BPH and 0.31±0.33 in PC. Both variants were expressed in all specimens, but expression of the PAP mRNA secreted variant was significantly decreased in PC (P<0.05).

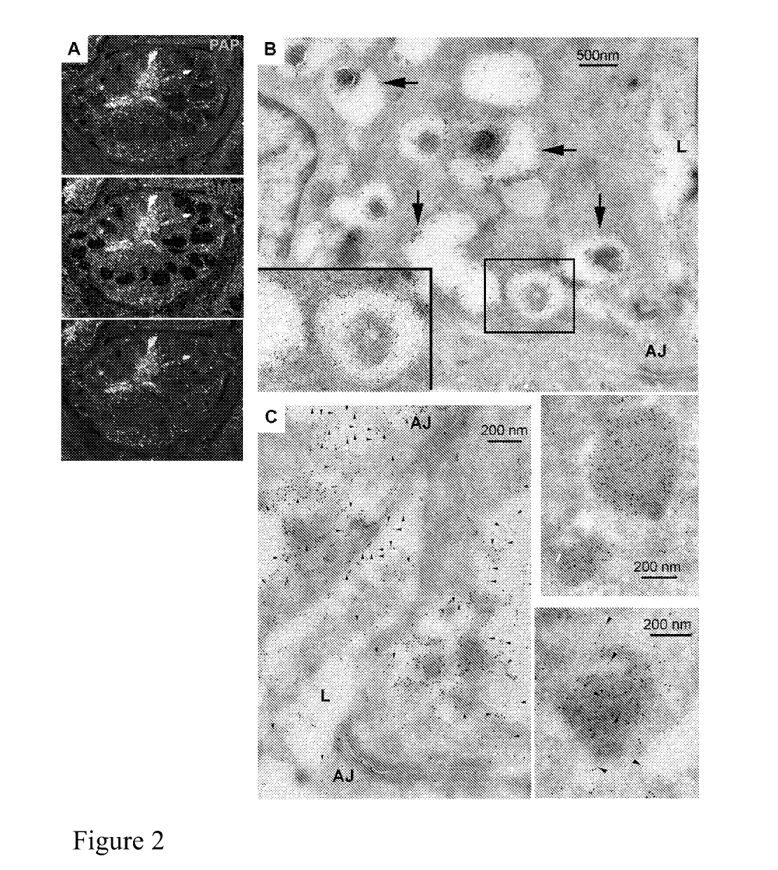

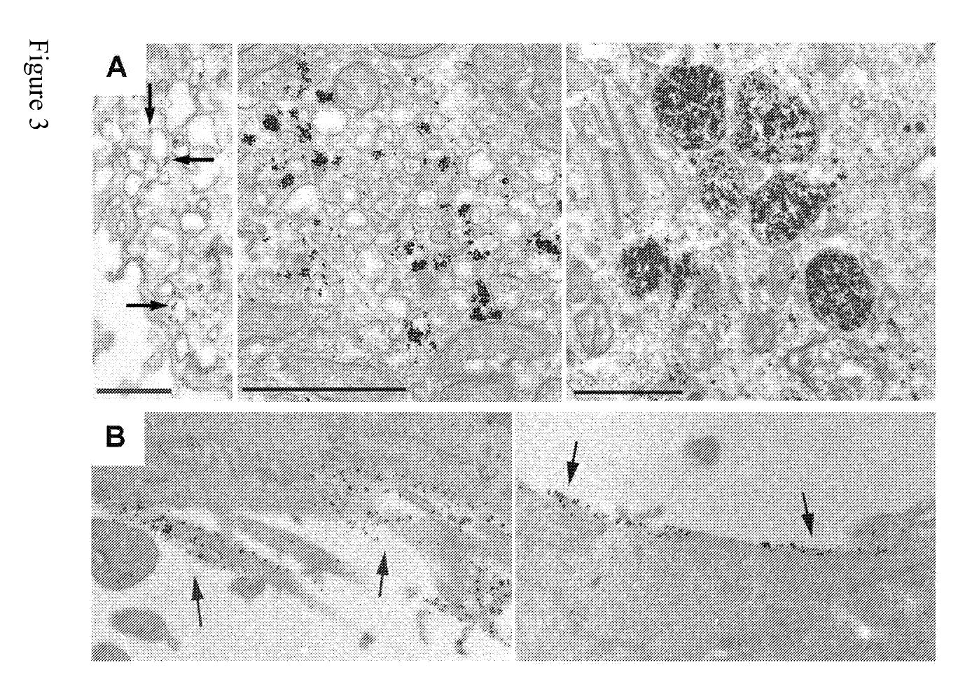

Immunofluorescence and electron microscopy were used to further investigate the localization of PAP in prostate cancer tissue. Immunofluorescence revealed that PAP is localized in vesicles located both in the basal and apical cytoplasm (FIG. 2A, PAP). In addition, strong labeling was observed in the lumen of the glands (FIG. 2A, PAP). PAP showed an almost complete colocalization with BMP (bis(monoacylglycero)phosphate) (FIG. 2A, BMP), a lipid highly enriched in late endosomes and multivesicular bodies (MVB) (FIG. 2A, merge). IEM was used to study the localization of PAP at higher resolution. PAP localized in the limiting and internal membranes of apical, electron-lucent vesicles (FIG. 2B, up), as well as in the internal membranes of MVBs (FIG. 2B, down). In agreement with the immunofluorescence labeling, PAP co-localized with BMP in all these structures (FIG. 2C, right), and PAP was observed in the membranous structures in the gland lumen (FIG. 2B, left). This suggests the luminal material may originate from PAP and BMP containing vesicles which have fused with the plasma membrane and emptied their contents into the lumen. These observations suggest that at least some luminal PAP is associated with intraendosomal membrane structures. Normally, the internal vesicles of the MVB are destined for degradation and are released into the lysosomal lumen. The residual membrane of the MVB is believed to retain those proteins which will escape degradation. MVB can also recycle to the plasma membrane and release their contents (exosomes) in vivo into the circulation.

### PAP is in Vesicles and Membranes

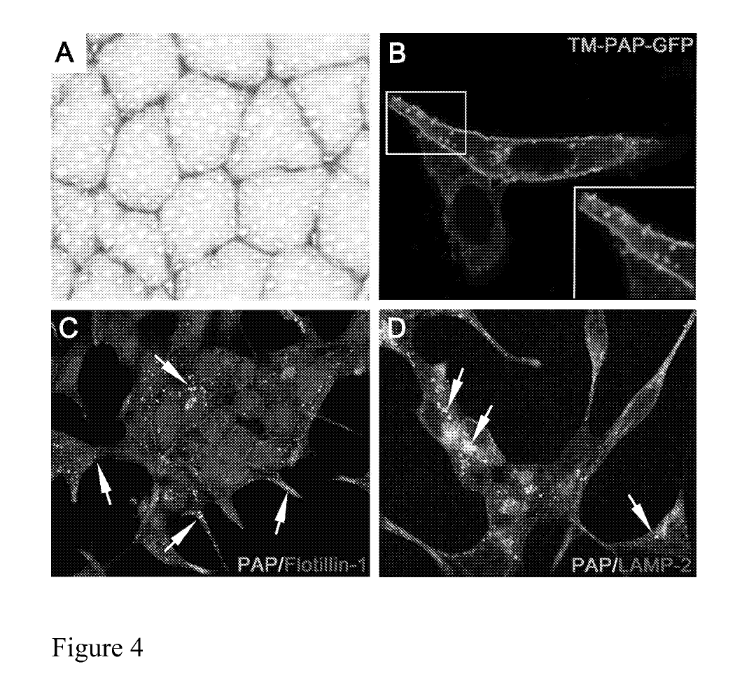

Similar pattern of distribution was detected in LNCaP cells, with a localization of PAP in endosome-like vesicles and lysosomes (FIG. 3A). The analysis of mouse Schwann cells showed PAP in the plasma membrane segments and filopodia-like structures (FIG. 3B). In the human skeletal muscle fibers, PAP expression was present in the sarcolemma (FIG. 4A).

The subcellular localization of TM-PAP-GFP in transfected PC-3 cells that do not express PAP endogenously was examined. Subcellular localization of TM-PAP-GFP in PC-3 cells was analyzed by confocal microscopy. TM-PAP-GFP was seen on the plasma membrane as expected and also observed in intracellular vesicles (FIG. 4B). In transfected control cells with an empty EGFP-N3 vector the signal was evenly distributed throughout the cells.

The co-localization of endogenous PAP with flotillin-1, which is associated with membrane lipid raft, was studied further and it showed that both proteins colocalize in plasma membrane and intracellular vesicles (FIG. 4C). Flotillins have been implicated in signaling, regulation of actin cytoskeleton and in membrane transport processes.

Considering the alignment results showing that PAP contains the lysosomal targeting sequence Yxxφ, the co-localization of PAP with a lysosomal associated type I membrane protein, LAMP-2 (FIG. 4D), was studied. The findings showed a clear co-localization between these proteins.

In conclusion, there are two widely expressed isoforms for PAP, a secreted and a transmembrane one. The importance of these two isoforms in prostate cancer development remains to be addressed, but the presence of a novel TM-PAP isoform in plasma membrane would suggest it to be involved in crucial cellular functions.

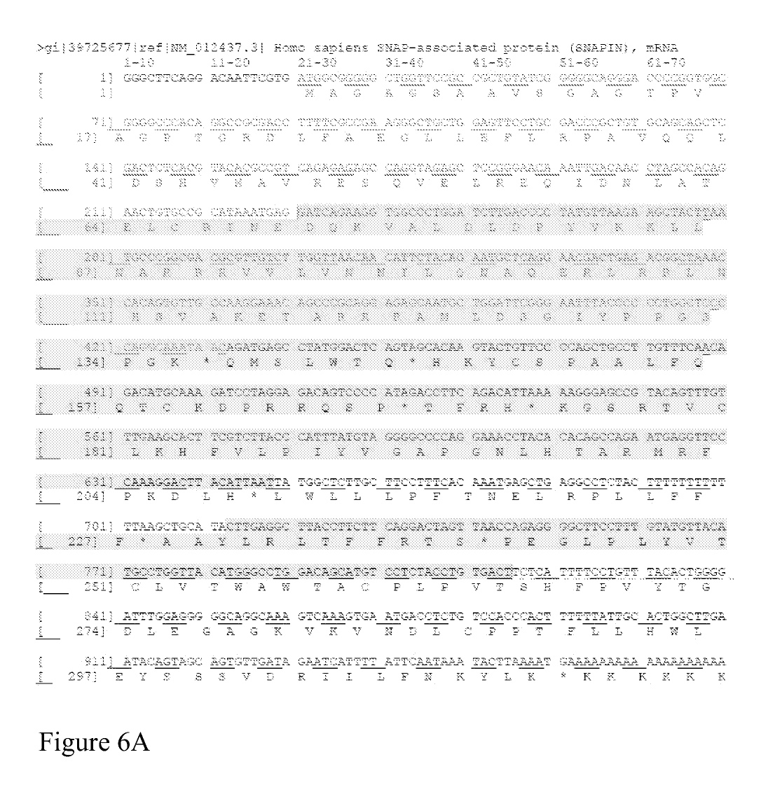

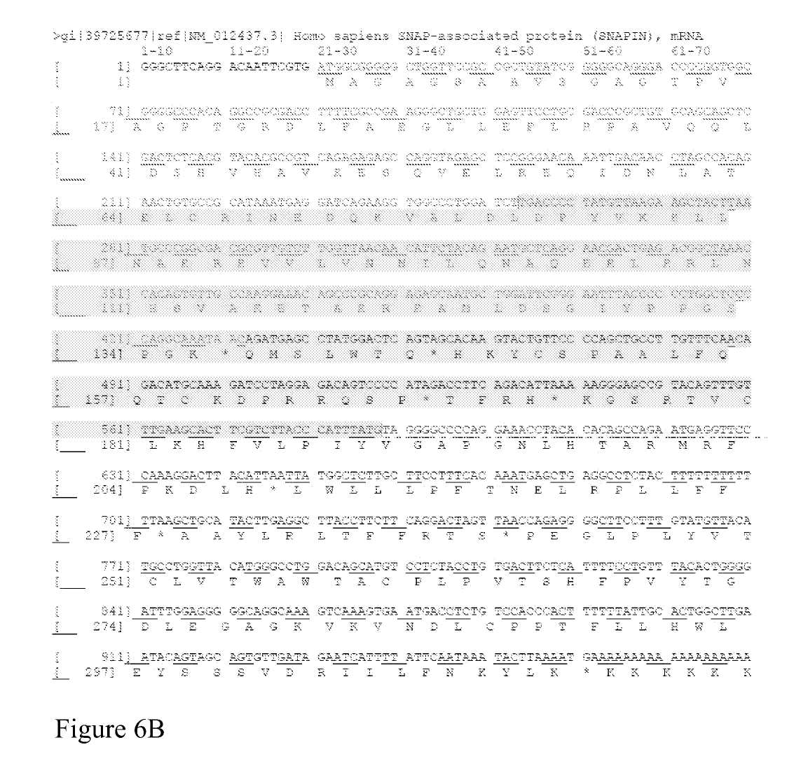

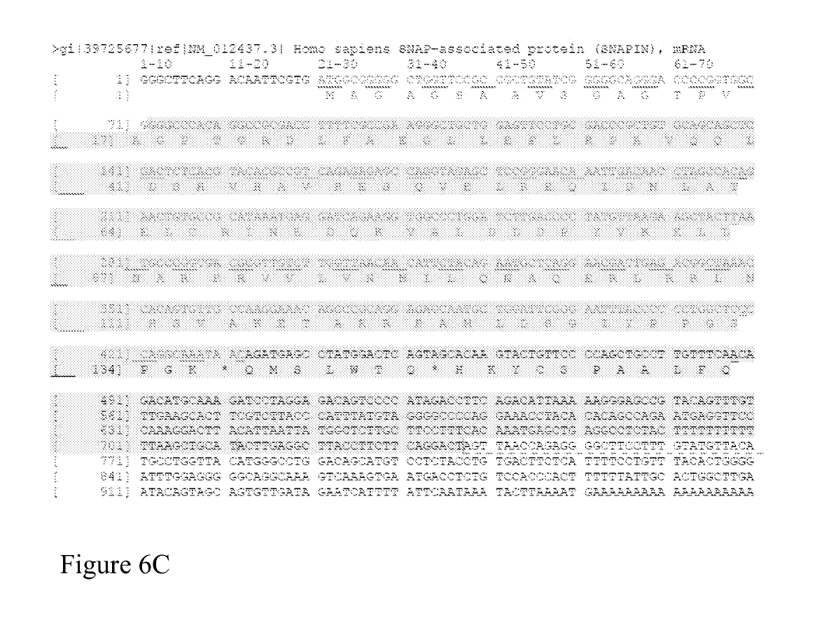

The yeast two-hybrid system was used to find out proteins interacting with TM-PAP. The yeast two-hybrid and co-immunoprecipitation was made as in “Snapin interacts with the Exo70 subunit of the Exocyst and modulates GLUT4 trafficking”, J Biol Chem 283, 324-331, 2008. The only finding was Snapin (FIG. 6 (A)-(C)), which is one of the proteins of exocytose mechanism. Therefore also TM-PAP is one part of the controlled secterion i.e. exocytose mechanism. In the microarray the most important group presented is the neurotransmission secretion mechanism. This mechanism is also in prostate and disruption thereof causes a change in the cell polarity. The apical microvilli will disappear, the ion channels, especially Cl and aquaporin channels, are downregulated, basal secretion decreases and cancer developes. Snapin is required together with TM-PAP in both the exocytosis and the endocytosis. TM-PAP will increase the production of cAMP and therefore also secretion. The regulation is via GPCR.

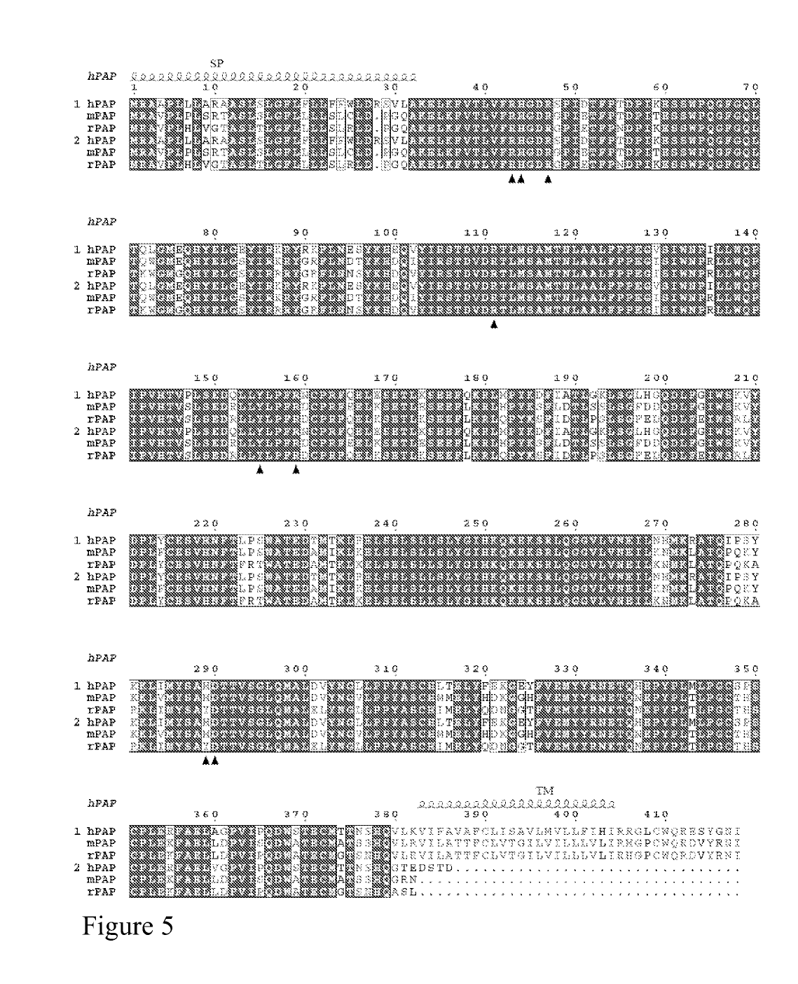

Thus, the present invention relates to a prostatic acid phosphatase protein having a transmembrane domain in the C-terminus, or the C-terminal part thereof. This protein may be used in any of the applications described herein. In one embodiment said transmembrane prostatic acid phosphatase protein is a human prostatic acid phosphatase protein. In still one embodiment said TM-PAP contains the amino acid sequence of SEQ ID NO: 1 or is a C-terminal fragment or a homologue thereof. Also the mouse and rat counterparts as shown in FIG. 5 are included. The about 25 kDa C-terminal part may also be cleaved off and used as a separate functional domain. The C-terminal part may be easily determined e.g. from FIG. 5 wherein several TM-PAP and secreted PAP sequences are aligned. The C-terminal part may contain e.g. the transmembrane region marked (amino acids 383-404).

TM-PAP may act in receptor transduction pathway together with G-protein coupled receptor (GPRC). The expression of RGSL2, a GPCR regulator, will increase greatly if PAP is knocked out or down. This fits to G(q/11) G-protein-mediated signaling. The secreted PAP may also regulate the extracellular part of TM-PAP, e.g. the binding of ligands to it, because the extracellular N-terminal part of TM-PAP is almost identical to the secreted PAP.

It was also discovered, that the knockout/knockdown of PAP induces RanGTPase and leads to increased and erroneous mitosis. This explains the prostate cancer observed in mice. Thus, PAP is a mitosis regulating factor. Therefore it may be used to treat any excess cell proliferation such as cancer, e.g. prostate cancer, lymphoproliferative disorders such as leukemia, bone marrow proliferation or other disorders.

One embodiment of the present invention relates to a nucleic acid molecule, such as a DNA or RNA molecule, encoding said TM-PAP of the invention or the C-terminal part thereof. Because of the degeneracy of the genetic code there are a number of different nucleic acid sequences encoding the TM-PAP of the invention. All such nucleic acid variants are in the scope of the present invention. Preferably said nucleic acid molecule is a DNA molecule.

One embodiment of the present invention relates to a replicable vector containing the nucleic acid molecule described above in operative association with an expression control sequence thereof. Such vector may be used for producing recombinant TM-PAP or the C-terminal part thereof of the present invention in a suitable host system.

The nucleic acid encoding the TM-PAP or the C-terminal part thereof of the invention may be inserted into said replicable vector for cloning or for expression. Various vectors are publicly available. The vector may, for example, be in the form of a plasmid, cosmid, viral particle or phage. The appropriate nucleic acid sequence may be inserted into the vector by a variety of procedures. In general, DNA is inserted into an appropriate restriction endonuclease site(s) using techniques well known in the art. Vector components may include for example one or more signal sequence(s), an origin of replication, one or more marker genes, an enhancer element, a promoter, and a transcription termination sequence. Construction of such suitable vectors containing one or more of these components employs standard ligation techniques which are well-known to a person skilled in the art.

Generally said TM-PAP or the C-terminal part thereof may be produced recombinantly by expressing in any suitable host cell, such as in a bacterial host cell. Such methods are well-known in the art and they are described in literature. It is essential that the protein is folded properly during the expression and it contains the necessary post-translational modifications.

One embodiment of the present invention provides the TM-PAP or the C-terminal part thereof of the invention for use as medicament. Still one embodiment of the present invention provides a pharmaceutical composition containing the TM-PAP or the C-terminal part thereof of the invention. Said composition may contain also other substances, such as pharmaceutically acceptable carriers, excipients or other pharmaceutical or therapeutical agents. Such compositions or formulations are well known to a person skilled in the art. One example of such medicament or pharmaceutical composition is a vaccine for any vesicle transport disorder, such as cancer, for example prostate cancer. Then medicament may be applied for example using methods disclosed in US2006/0294615, wherein the TM-PAP or the C-terminal part thereof may be in a liposome, which may be comprised of lipofectin, or the TM-PAP or the C-terminal part thereof is administered as coupled to a monoclonal antibody, which may be immunologically specific to a cancer cell, such as human prostate cancer cell.

One embodiment of the present invention provides the use of the prostatic acid phosphatase protein or the C-terminal part thereof of the invention for manufacturing medicament for treating vesicle transport disorders, such as cancer, for example prostate cancer, or other metabolic disorders related to e.g. glucose or lipid metabolism.

One embodiment of the present invention provides a method for treating vesicle transport disorders, such as metabolic disorders related to e.g. glucose or lipid metabolism by administering the TM-PAP or the C-terminal part thereof of the invention to a patient suffering from said disorder. One embodiment of the present invention provides a method for treating vesicle transport disorders related to cancer by administering the TM-PAP or the C-terminal part thereof of the invention to a patient suffering from said disorder, such as cancer, such as prostate cancer. TM-PAP may be administered by any suitable means, for example by methods disclosed in US2006/0294615, such as by administering a nucleic acid comprising the coding sequence of TM-PAP to the carcinoma and allowing the coding sequence to be expressed in the carcinoma, or it may be administered as coupled to a monoclonal antibody, which may be immunologically specific to the cancer cell.

Still one embodiment of the present invention provides a method for treating vesicle transport disorders such as metabolic disorders related to e.g. glucose or lipid metabolism by induction of TM-PAP to restore the physiological condition. Still one embodiment of the present invention provides a method for treating vesicle transport disorders such as cancer by induction of TM-PAP to restore the physiological condition.

One embodiment of the present invention provides a method for delivering drugs, nanoparticles, imaging reagents or other useful molecules to cells using said transmembrane prostatic acid phosphatase protein as a carrier or vehicle. For example the drugs nanoparticles, imaging reagents or other useful molecules may be attached to an antibody binding TM-PAP which then finds its way to the appropriate cells. The secreted form of PAP is not able to do this. In one embodiment the antibody binds the extracellular part of TM-PAP. The antibody may also be labeled with any suitable detectable label, such as radioactive or fluorescent label, and it can be used for example to detect metastases. In one embodiment nanoparticles containing drugs have such antibody on their surface.

One embodiment of the present invention provides a method for diagnosing vesicle transport disorders, such as cancer, such as prostate carcinoma, by quantitating TM-PAP and secreted PAP in a tissue and determining the ratio thereof. As the expression of secreted PAP variant mRNA is decreased in cancer, the elevated ratio of TM-PAP:secreted PAP may indicate the cancer. The ratio may be determined from a tissue sample by any suitable means known for a person skilled in the art enabling the quantitation of TM-PAP and secreted PAP, such as quantitative real-time PCR. U.S. Pat. No. 7,094,533 discloses methods for diagnosing androgen-insensitive prostate carcinomas. In said methods the cellular PAP protein is quanti-fied by an antibody immunologically specific to the cellular PAP or the concentration of cellular PAP mRNA is quantified by PCR, Northern or Southern blot. Said methods may be applied also for quantifying the ratio of TM-PAP and secreted PAP.

“Knockout” as used herein refers to a process of purposely removing a particular gene or trait from an organism or cell. Generally a knockout is a site-specific integration that usually deletes an essential part of a gene of interest. Methods of making knockouts are generally known in the art, for example microinjection and targeted mutation methods. The knockouts of the present invention may be done with any known knockout methods, such as any heritable modifications of the PAP gene, as long as they result to substantial disruption of at least part of the PAP gene or an expression regulation region thereof resulting in substantial disruption of the original function of expressed PAP enzyme or the original function of PAP gene. In one embodiment the knockout is done by replacing a part of the PAP gene with an external nucleic acid molecule and introducing this modified gene into the genome of the animal to replace the original PAP gene. The knockout may also be done by removing the whole gene of interest.

Gene “knockdown” refers to techniques by which the expression of one or more of an organism's genes is reduced, either through genetic modification (a change in the DNA of one of the organism's chromosomes) or by treatment with a reagent such as a short DNA or RNA oligonucleotide with a sequence complementary to either an mRNA transcript or a gene. If genetic modification of DNA is done, the result is a “knockdown organism” (Wikipedia).

The terms “substantially disrupted” or “substantially reduced” are herein intended to mean that substantially lower amount of normal PAP gene product is produced in cells or in organism when compared to normal cells or organisms. Preferably such lower amount refers to essentially undetectable amount of normal gene product. This type of mutation is generally called as a “null mutation” and an animal carrying such a mutation is also referred to as a “knockout animal”. Correspondingly, when an animal cell is used the cell carrying such a mutation is referred to as “knockout cell”.

The “decrease in activity” of the PAP as used herein refers to a decrease of total enzyme activity of said disrupted or substantially disrupted PAP gene product in certain cells. Said decrease in activity may be partial or the activity may be totally lost and it is sufficient to cause disorders related to PAP gene, such as described in the specification. Said decrease in activity may relate for example to reduced level of expressed PAP, for example if the promoter region of the PAP gene has been altered, or to disrupted structure or function of PAP enzyme. “Decrease in the activity of (expressed) PAP” refers not only to expressed PAP which has lower activity compared to normal PAP, but also to PAP which is expressed at lower levels than normally. This embraces also PAP which is substantially not expressed at all.

The “PAP gene” as used herein refers to any suitable (transmembrane) prostatic acid phosphatase gene or homologue or derivative thereof. The PAP gene to be used in the present invention may be of any suitable origin, plant or animal, such as rat, mouse or human PAP gene. In one embodiment the human prostatic acid phosphatase (hPAP) gene is used to provide a model for investigating human PAP-related disorders. The human PAP gene may be inserted to another species, such as rat or mouse, or to a cell thereof, to provide a transgenic animal or cell model for investigating said disorders or diseases. Also PAP genes of other origins may be used, such as mouse PAP (mPAP) or rat PAP (rPAP). Generally any PAP gene from any suitable organism, which will produce, when expressed in a cell, a substantially functional PAP enzyme, may be used. Such PAP gene may be a homologue of a known PAP gene from certain species having insertions or deletions of amino acids, but still having sufficient homology with the original gene to produce substantially analogous PAP enzyme. Generally such homology is preferably at least 50%, more preferably 80% and most preferably 90% at amino acid level. The “fragment” of TM-PAP refers to a C-terminal fragment having the transmembrane part.

“Vesicle transport disorder” as used herein refers to a disorder wherein the function of the transport mechanism of the cell is disrupted (trafficking disease). This can also be called an exocytosis and/or endocytosis disorder which may lead to several diseases, such as cancer. In this mechanism involved are so called SNARE complexes. The primary role of SNARE proteins is to mediate fusion of cellular transport vesicles with the cell membrane at the porosome or with a target compartment (such as a lysosome). SNARE contains ia. the Snapin protein which is regulated by TM-PAP as described herein.

“Phosphatidylinositol phosphate” (PIP) refers to any phosphatidylinositol phosphate as described herein, such as phosphatidylinositol 3-phosphate PI(3)P, phosphatidylinositol 4-phosphate PI(4)P, PI(5)P, PI(3,5)₂, PI(4,5)P₂, PI(3,4,5)P₃ or corresponding soluble inositol phosphate (IP).

The present invention also utilizes knockout/knockdown animals and cells wherein at least one allele of an endogenous transmembrane prostatic acid phosphatase (TM-PAP) gene is functionally disrupted in somatic and/or germ cells. Said animals or cells may be heterozygous or, preferably, homozygous for the TM-PAP knockout/knockdown.

Animals to be used in the animal model of the present invention include any suitable non-human animals, such as vertebrates, or more particularly mammals. The term animal includes an individual animal in all stages of development, including embryonic and fetal stages. In one embodiment of the invention the animal is a rodent, such as mouse or rat, which are generally used as similar applications may be adapted to both species. The cells to be used include any suitable cells, from plants or animals, for example ones derived from the animal described above or human cell lines.

The disruption may be made to exon 3 of the TM-PAP gene. This is preferred because exon 3 is involved with TM-PAP activity. This will ensure that the activity of the TM-PAP enzyme will be abolished. Said exon 3 may be knocked out totally or its function may be decreased partially. However, similar disruptions or knockouts may be also made to another suitable part of TM-PAP gene, for example by introducing deletions or insertions of nucleic acids to obtain the defective TM-PAP. The TM-PAP gene may also be totally removed. Said disruption may also refer to the level of expressed gene product.

Said disruption may be introduced into said transmembrane prostatic acid phosphatase gene by replacing at least part of it with an external nucleic acid sequence. One example of such external nucleic acid sequence is the commonly used neo cassette.

Said transmembrane prostatic acid phosphatase gene may be originated from different species, such as human, mouse or rat prostatic acid phosphatase gene or homologue thereof, or it is a recombinant TM-PAP gene.

Said external nucleic acid molecule used for replacing part of the TM-PAP gene may be any suitable nucleic acid molecule containing suitable nucleic acid sequence which is able to decrease the activity or level of expressed prostatic acid phosphatase when inserted to the TM-PAP gene, for example at the location of exon 3. Targeting to generate a null or mutated allele is usually accomplished by insertion of a selectable marker into a gene causing disruption of splicing, promoter function, or reading frame, with or without deletion of some of the gene. One commonly used selectable marker gene for making knockouts is the neo gene, which confers resistance to the antibiotic neomycin. Also other suitable sequences may be used.

One embodiment of the present invention provides methods for testing and screening a compound, such as a drug candidate, for a therapeutic effect, comprising: administering said compound to a cell or a non-human animal having disruption in the prostatic acid phosphatase (PAP) gene or regulation thereof resulting in a decrease or absence of the activity or the level of prostatic acid phosphatase, and determining if said compound substantially restores the unbalanced phosphatidylinositol phosphate cascade and/or signaling pathway related to PAP expression or activity on said cell or said animal, said restoring indicating said compound being therapeutically effective for treating vesicle transport disorders or disorders related to unbalanced phosphatidylinositol phosphate cascade and/or signaling pathway.

In one embodiment of the present invention said restoring is decrease in PI(4,5)P₂ accumulation. In another embodiment of the present invention said restoring is decrease in the level of PI(3)P.

In one embodiment of the present invention the response on a similar animal or cell not exposed to said compound is determined and the responses are compared in order to find out if there is an effect for said compound.

Such control animal may be exposed for example to placebo or other compound. In one embodiment the response of a knockout/knockdown animal or cell is compared to one of a wild type animal. In other embodiments, the knockout/knockdown animals or cells are examined directly without comparison to a wild-type animal. Such methods are generally well known in the art. Said therapeutic effect is against a disorder related to transmembrane prostatic acid phosphatase, such as any vesicle transport disorder. Non-limiting examples of such diseases or disorders are prostatic atypical hyperplasia, prostatic intraephitelial neoplasia, carcinoma; prostatitis, respiratory or kidney diseases; or inflammatory, immunodefence or autoimmunediseases and other disorders related to phosphorylation/dephosphorylation or transport of phosphatidylinositol 3-phosphate (PI(3)P). As TM-PAP is now known to reduce the amount of PI(3)P, said disorder may be associated with the PI(3)P metabolism and related mechanisms, such as insulin response, lipid metabolism, growth factor response, cell division, apoptosis etc., which are regulated by PI(3)P. Said diseases and disorders include prostate, bladder and pancreatic cancer, myopathy (myodegeneration) and neuropathy (neurodegeneration). A compound found with the method of the invention may be used as medicament for treating the disorders disclosed herein. Therefore, one embodiment of the present invention provides a therapeutic compound obtained with the method on any of the preceding claims. Non-limiting example of such compound may be a compound acting as an enhancer for the expression of TM-PAP.

One embodiment of the present invention provides methods for treating said diseases or disorders related to TM-PAP by gene therapy and other methods to restore the activity of TM-PAP. A compound affecting to the expression or effect of TM-PAP may be administered to tissues or cells of a patient suffering a condition or disorder related to TM-PAP, as described above. Also gene therapy affecting to or restoring the activity of TM-PAP may be given to such patient. The gene therapy preferably restores at least part of the TM-PAP activity. Suitable genes to be used in such gene therapy include any suitable TM-PAP gene or nucleotide vector of the invention as described herein. The gene therapy or other treatment may be administered to the patient for example by injection to target, such as to prostate or by prostate artery, as a virus vector. Such gene therapy methods are generally well known in the art and a person skilled in the art can choose a suitable method for the treatment. Examples of commonly used virus vectors which may be used to carry the TM-PAP gene include baculo, adeno and lenti viruses (see e.g. Airenne et al.: “Baculovirus-mediated Gene Transfer: An Evolving New Concept”, Templeton N S, ed. Gene and Cell Therapy, 181-197, New York, Marcel Dekker, 2004; Kost et al.: “Baculovirus as versatile vectors for protein expression in insect and mammalian cells, Nature Biotechnology 2005:23, 567-575).

To find out the physiological function of PAP in the prostate PAP knockout (KO) mouse model was generated by deleting the 3^{rd} exon of the mPAP gene. The present inventor found out that PAP is PI(3)P-lipid phosphatase, regulates PI(3)P membrane traffic, and its inactivation is causative for prostate cancer. The corresponding publication (Vihko P T, Quintero I, Ronka A E, Herrala A, Jantti P, Porvari K, Lindqvist Y, Kaija H, Pulkka, A, Vuoristo J, et al. 2005 Prostatic acid phosphatase (PAcP) is PI(3)P-phosphatase and its inactivation leads to change of cell polarity and invasive prostate cancer. p1328 (#5239) Proceedings of the AACR: 96th Annual meeting, Anaheim, Calif.) is incorporated herein by reference. Materials, methods, experiments and discussion for preparing a PAP knockout mouse and testing thereof are described in WO06051172A1. As the TM-PAP is encoded by the same gene as PAP, said PAP animal model can be directly applied to TM-PAP the applications thereof.

### Experimental Procedures

### Antibodies

Lysosomal associated membrane protein (LAMP) 2 monoclonal antibody was developed by J. T. August and J. E. K. Hildreth (Johns Hopkins University) and obtained from the Developmental Studies Hybridoma Bank (developed under the auspices of the NICHD and maintained by The University of Iowa, Department of Biological Sciences, Iowa City, Iowa 52242). Mouse monoclonal antibody against bis(monoacylglycero)phosphate (BMP) was a generous gift from Prof. Jean Gruen berg (University of Geneva). PAP polyclonal antibody was generated by the present inventor's group. Flotillin-1 was from BD Biosciences.

### PCR-Based Cloning of Novel PAP Variants

To obtain cDNA encoding rat TM-PAP, total RNA isolated from rat prostate was amplified by RT-PCR using primers 5′-ACCATGAGAGCTGTCCCTCTG-3′ and 5′-TCAGATGTTCCGATACAC-3′ designed from the rat genomic region which was found in silico based on inventor's hypothesis of high similarity in rat and mouse PAP gene structure and the peptide sequence of a possible TM domain.

To obtain a cDNA encoding human TM-PAP, RT-PCR was performed using total RNA from human prostate as a template and primers 5′-ATGAGAGCTGCACCC-3′ and 5′-GCTCTGGGCAGATTCAAAAGG-3′.

The resulting RT-PCR products were cloned into a pCRll-TOPO vector (Invitrogen) and sequenced.

### Splice Variant-Specific RT-PCR

Reverse transcription of total RNA to cDNA and subsequent amplification were performed using the GeneAmp RNA PCR kit (Applied Biosystems) according to the manufacturer's instructions. Total RNA was isolated from human and mouse tissue specimens, mouse fibroblasts, SW10, LNCaP and PC-3 cells, and used as a template. Primers are described in supplementary data.

Quantitative real-time RT-PCR to amplify cDNA fragments for the individual PAP variants in human prostate tissue specimens was performed by using TaqMan chemistry on an ABI Prism 7700 sequence detection system (Applied Biosystems). Human tissue specimens were obtained as redundant material for diagnosis from hormonally untreated patients going through transurethral resection for benign prostatic hyperplasia (BPH) or total prostatectomy in the case of prostate cancer (PC) in the Oulu University Hospital. Histopathology was confirmed and cancer tissue dissected by the pathologist. The cDNA was synthesized from 0.5 μg total RNA derived from frozen prostate tissue samples, BHP (n=27) and PC (n=19). Primers are described in supplementary data. TM-PAP mRNA variant, PAP mRNA secreted variant, and 18S RNA levels were measured. The results were normalized to 18S quantified from the same samples. Statistical analyses were performed with Student's two-tailed t-test. Difference was considered significant if P<0.05.

The permissions to use the human (prostate and muscle) and mice specimens for research purposes have been obtained from Oulu University Hospital's Ethical Committee and National Authority for Medicolegal Affairs, and from The Animal Experimentation Committee, University of Oulu, Finland, respectively.

### Cell Culture

The human prostate carcinoma cell lines PC-3 and LNCaP, and mouse neuronal Schwann cells (SW-10) were obtained from American Type Culture Collection (ATCC) and grown according to the instructions of ATCC.

Mouse skin fibroblasts were obtained by culturing of two-day-old mouse skin explants and subcultured.

### Immunofluorescence and Confocal Microscopy

Immunolabeling of human samples was performed according to Andrejewski et al. (Andrejewski N, Punnonen E L, Guhde G, et al. Normal lysosomal morphology and function in LAMP-1-deficient mice. J Biol Chem 1999; 274: 12692-701). Samples were fixed in 4% paraformaldehide/2.5% sucrose, mounted on sample holders and frozen in liquid nitrogen. Cryosections for fluorescence and electron microscopy were cut at −100° C. Fluorescent (Alexa Fluor 594 goat anti-mouse IgG and Alexa Fluor 488 goat anti-rabbit IgG, Molecular Probes) or gold-conjugated (5-nm gold goat anti-mouse IgG and 10-nm goat anti-rabbit IgG, British Bio Cell, Gardiff) secondary antibodies were used. The samples were analyzed using a confocal microscope (Leica Microscopy and Scientific Instruments Group) or a Jeol 1200 EX electron microscope (Tokyo).

LNCaP and SW-10 cells grown on Thermanox plastic coverslips (Nalgene-Nunc, Rochester, N.Y.) were subjected to pre-embedding immunolabelling with anti-PAP antibody (Vihko P, Sajanti E, Jänne O, Peltonen L, Vihko R. Serum prostate-specific acid phosphatase: development and validation of a specific radioimmunoassay. Clin Chem 1978; 24: 1915-9) as described earlier (Uchiyama K, Jokitalo E, Kano F, et al. VCIP135, a novel essential factor for p97/p47-mediated membrane fusion, is required for Golgi and ER assembly in vivo. J Cell Biol 2002; 159: 855-66) and processed for epon embedding. Sections were cut parallel to the coverslip, post-stained with uranyl acetate and lead citrate, and imaged with Tecnai 12 (FEI Corp.) electron microscope at 80 kV.

LNCaP and PC-3 cells grown on cover slips were fixed with 4% paraformaldehyde, and permeabilized with blocking buffer (1% BSA/0.2% saponin in PBS). Primary antibodies were incubated for 1 hour at room temperature. Fluorescent second antibodies were used (Alexa Fluor 488 goat anti-rabbit IgG (Molecular Probes) and TRITC goat anti-mouse IgG (Sigma)), and 4,6-diamidino-2-phenylindole (DAPI, Fluka chemicals) to stain the nucleus. Cells were embedded in Immuno Mount (Thermo Sandon). Confocal imaging was performed using Olympus FluoView 1000 confocal microscope.

Transfection of PC-3 Cells with TM-PAP-GFP

cDNA for human TM-PAP was obtained by RT-PCR using human prostate total RNA as a template and primers as follows: 5′-TTAGGATCCACCATGAGAGCTGCACC-3′ and 5′-AATGGATCCGATGTTCCCATAGGATTC-3′. The PCR product was cloned into pCR 2.1-TOPO plasmid (Invitrogen). From the recombinant plasmid, the BamHI restriction fragment containing the coding region of TM-PAP was cloned into a pEGF-N3 (BD Biosciences Clontech) plasmid. Restriction digestions and sequencing confirmed the direction of the insert and sequence of the construction. PC-3 cells were grown to 80-90% confluence and transfected using Lipofectamine 2000 (Invitrogen) according to manufacturer. Transfection (DNA:Lipofectamine, 1:2) was performed for 5 hours at 37° C. in Opti-MEM medium (Gibco). Cells were incubated in normal growth medium for 24 hours at 37° C. before experiments.

### Immunohistochemistry

Histopathologically normal skeletal muscle biopsies were chosen for the study (from the files of the pathology laboratory, Oulu University Hospital, Finland). The muscle biopsies were orientated and mounted in optimum cutting temperature (OCT) compound, frozen in isopenthane and liquid nitrogen, and cut for immunohistochemistry (10 μm). The immunohistochemistry procedure was performed using an anti-PAP antibody (1:500) (Vihko P, Sajanti E, Jänne O, Peltonen L, Vihko R. Serum prostate-specific acid phosphatase: development and validation of a specific radioimmunoassay. Clin Chem 1978; 24: 1915-9), the EnVision (DAKO) staining kit and diaminobenzidine as chromogen (DAKO).

This invention has been described with an emphasis upon some of the preferred embodiments and applications. However, it will be apparent for those skilled in the art that variations in the disclosed embodiments can be prepared and used and that the invention can be practiced otherwise than as specifically described herein within the scope of the following claims.

1. A pharmaceutical composition containing a prostatic acid phosphatase protein having a transmembrane domain and endosomal/lysosomal targeting signal in the C-terminus, or the C-terminal part thereof.

2. The pharmaceutical composition of claim 1, wherein the prostatic acid phosphatase protein is a human prostatic acid phosphatase.

3. The pharmaceutical composition of claim 1, wherein the prostatic acid phosphatase protein has the amino acid sequence of SEQ ID NO: 1 or is a C-terminal fragment or a homologue thereof.

4. (canceled)

5. The method of claim 10, wherein the prostatic acid phosphatase protein is a human prostatic acid phosphatase.

6. The method of claim 10, wherein the prostatic acid phosphatase protein has the amino acid sequence of SEQ ID NO: 1 or is a C-terminal fragment or a homologue thereof.

7-9. (canceled)

10. A method for treating vesicle transport disorders, such as metabolic disorders related to glucose or lipid metabolism, or disorders related to cancer, such as prostate cancer, by administering a prostatic acid phosphatase protein having a transmembrane domain and endosomal/lysosomal targeting signal in the C-terminus, or the C-terminal part thereof to a patient suffering from said disorder.

11. The method of claim 10, wherein the step of administering a prostatic acid phosphatase protein or the C-terminal part thereof is includes administering a nucleic acid comprising the coding sequence of thereof to the carcinoma and allowing the coding sequence to be expressed in the carcinoma.

12. The method of claim 10 wherein the transmembrane prostatic acid phosphatase protein or the C-terminal part thereof is administered in a liposome.

13. The method of claim 12, wherein the transmembrane prostatic acid phosphatase protein or the C-terminal part thereof is administered coupled to an antibody specific to a cancer cell.

14. A method for delivering drugs, nanoparticles, imaging reagents, such as detectable labels, or other molecules to cells using a prostatic acid phosphatase protein having a transmembrane domain and endosomal/lysosomal targeting signal in the C-terminus, or the C-terminal part thereof as a carrier comprising attaching the detectable labels or other molecules to the prostatic acid phosphatase protein or the C-terminal part thereof.

15. The method of claim 14, wherein the prostatic acid phosphatase protein is a human prostatic acid phosphatase.

16. The use of claim 14, wherein the prostatic acid phosphatase protein has the amino acid sequence of SEQ ID NO: 1 or is a C-terminal fragment or a homologue thereof.

17. The method of claim 14, wherein the detectable labels or other molecules include the drugs attached to an antibody binding capable of binding the prostatic acid phosphatase protein or the C-terminal part thereof.

18. The method of claim 14, wherein the detectable labels or other molecules include nanoparticles containing drugs and having an antibody on their surface capable of binding the prostatic acid phosphatase protein or the C-terminal part thereof.

19-34. (canceled)

35. The method of claim 15, wherein the detectable labels or other molecules include drugs attached to an antibody binding capable of binding the prostatic acid phosphatase protein or the C-terminal part thereof.

36. The method of claim 16, wherein the detectable labels or other molecules include drugs attached to an antibody binding capable of binding the prostatic acid phosphatase protein or the C-terminal part thereof.

37. The method claim 15, wherein the detectable labels or other molecules include nanoparticles containing drugs and having an antibody on their surface capable of binding the prostatic acid phosphatase protein or the C-terminal part thereof.

38. The method claim 16, wherein the detectable labels or other molecules include nanoparticles containing drugs and having an antibody on their surface capable of binding the prostatic acid phosphatase protein or the C-terminal part thereof.
